(19) United States
(12) Patent Application Publication
(10) Pub. No.: US 2013/0237900 A1
(43) Pub. Date: Sep. 12, 2013

(54) OPHTHALMIC SURGICAL SYSTEM AND CONTROL APPARATUS THEREFOR

(71) Applicant: CARL ZEISS MEDITEC AG, Jena (DE)

(72) Inventor: Christoph Hauger, Aalen (DE)

(73) Assignee: Carl Zeiss Meditec AG, Jena (DE)

(21) Appl. No.: 13/854,601

(22) Filed: Apr. 1, 2013

Related U.S. Application Data
(63) Continuation of application No. PCT/DE2011/001778, filed on Sep. 24, 2011.

(30) Foreign Application Priority Data
Sep. 30, 2010 (DE) 10 2010 047 010.4

(51) Int. Cl. A61F 9/007

(52) U.S. Cl. CPC A61F 9/00745
USPC 604/22

(57) ABSTRACT

A control apparatus for an ophthalmic surgical system includes a measuring device with which, at a first time point and a second time point, the volume of an anterior chamber of the eye of an eye to be treated by phacoemulsification is determined. A control unit, dependent on the determined volume at the first time point and at the second time point, calculates a control variable to control a pressure or a volumetric flow rate in an irrigation line and/or an aspiration line of the ophthalmic surgical system.

### CROSS REFERENCE TO RELATED APPLICATIONS

This application is a continuation application of international patent application PCT/DE2011/001778, filed Sep. 24, 2011, designating the United States and claiming priority from German application 10 2010 047 010.4, filed Sep. 30, 2010, and the entire content of both applications is incorporated herein by reference.

### FIELD OF THE INVENTION

The invention relates to a control apparatus for an ophthalmic surgical system for controlling pressure or volumetric flow rate in an irrigation line or an aspiration line, and also to an ophthalmic surgical system with such a control apparatus.

### BACKGROUND OF THE INVENTION

There are a number of surgical techniques for treating clouding within the eye lens, which is referred to as a cataract in medicine. The roost common technique is phacoemulsification, in which a thin needle is introduced into the diseased lens and excited to vibrate by means of ultrasound. The vibrating needle emulsifies the lens in the direct vicinity thereof in such a manner that the created lens particles can be suctioned away through a line via a pump. In the process, a rinsing fluid (irrigation fluid) is supplied, with the particles and the fluid being suctioned away through an aspiration line. Once the lens has been completely emulsified and removed, a new artificial lens can be inserted into the empty capsular bag, and so a patient treated in this way regains good visual acuity.

Approximately 600,000 operations of this type are carried out in Germany each year, with such an intervention harboring a relatively low rate of complications. However, such an operation still requires the treating surgeon to have much experience. Although the vibrating needle can break up an eye lens into small particles in a relatively reliable fashion, these particles have different sizes. If the particle is smaller than the internal diameter of an aspiration line, which is usually routed within the vibrating needle, such a small particle can he suctioned away without problems together with the associated fluid. However, if the particle is larger than the smallest internal diameter of the aspiration line, it either does not even enter the line or blocks the same. The blocked-line state is referred to as an occlusion. The occlusion results in strong negative pressure in the aspiration line. If the particle then breaks into smaller parts, for example as a result of a more vigorous vibration of the needle, such that the occlusion breaks up, strong suction is created in the region of the needle tip. In the process, a wall of a lens capsular bag can be suctioned to the needle tip and pierced by the needle. The capsular bag being pierced, leads to significant complications for the patient; these must foe avoided at all costs. During an operation, the treating surgeon must therefore be very attentive in order to avoid damage to the patient's eye. However, an operation takes a relatively long period of time if work has to be undertaken with so much care. The breakup of an occlusion moreover not only has an effect on the aspiration line, but also affects the irrigation line. There can also foe strong pressure variations therein if a blockage in the aspiration line suddenly breaks up.

### SUMMARY OF THE INVENTION

It is an object of the invention to provide a control apparatus for an ophthalmic surgical system, via which a pressure or a volumetric flow rate in an aspiration line or an irrigation line can be controlled in a quick and reliable manner, such that there is a low risk of damaging a patient's eye, wherein, nevertheless, the surgical procedure can be performed quickly. It is furthermore an object of the invention to provide an ophthalmic surgical system which is equipped with such a control apparatus.

The control apparatus according to the invention for an ophthalmic surgical system includes:
a measuring device via which it is possible, at a first time and a second time, to determine the volume of an anterior chamber of the eye of an eye to be treated by phacoemulsification; and,
a control unit which, dependent on the determined volume at the first time and at the second time, calculates a control variable via which it is possible to control a pressure or a volumetric flow rate in an irrigation line and/or an aspiration line of the ophthalmic surgical system.

At the start of an occlusion, or when the occlusion breaks up, there is a change in the pressure in the aspiration line or irrigation line. This can be determined by a pressure measuring instrument, which is coupled to the irrigation line or aspiration line. A disadvantage is that a relatively long time passes before a pressure change in the eye is captured by a pressure measuring device outside of the eye, that is, for example, in a console of an ophthalmic surgical system. This means that only relatively sluggish open-loop or closed-loop control of the pressure or volumetric flow rate in the aspiration line and/or irrigation line is possible, and so injury to the patient's eye cannot be avoided despite the surgeon taking great care.

The invention proceeds from the idea of capturing directly in the eye the results of a pressure change at the start of an occlusion or at the breakup of an occlusion. Hence it is not necessary to establish a change in a surgical parameter outside of the eye. If a measuring device captures the volume of an anterior chamber of the eye at a first time and at a second time, it is possible, for example, to calculate a difference between the volume existing at the first time and the volume existing at the second time. If the value of the difference differs from zero, this indirectly provides a statement in respect of a pressure change in the anterior chamber of the eye, without, a pressure having to be measured. A strongly changing volume in the anterior chamber of the eye can therefore be evaluated in such a way that surgical parameters or control variables that help to reduce injury to a patient's eye are influenced. Hence, it is no longer necessary for the surgeon to minimize the effects in the case of a sudden breakup of an occlusion with uttermost attention. If an occlusion occurs, a control unit of the ophthalmic surgical system can, on the basis of the volume measurement at the first time and at the second time, be controlled in such a manner that such a strong pressure change in the aspiration line no longer occurs during the breakup of the occlusion. Since this can occur in a faster and more efficient fashion via an electronic control than, for example, by means of a manual control of a parameter by means of a foot pedal, a phacoemulsification can be carried out in a shorter period of time and with greater safety by the control apparatus according to the invention.

The measuring device preferably has an optical system such as, for example, an OCT system for measuring a three-dimensional object region. An OCT system, for example with a rotating or scanning fiber, renders it possible to capture a region below the cornea, above the iris and above the lens capsular bag. If a strong change in pressure announces itself, this can be established indirectly by a change in volume in the anterior chamber of the eye.

The measurement accuracy can be increased if the measuring device can be used to establish the volume of the needle of a phacoemulsification handpiece within the anterior chamber of the eye. During an operation, the vibrating needle in the anterior chamber of the eye is moved, on occasion, into the edge region of the eye lens or into the central region of the eye lens. In doing so, the volume required and displaced in the anterior chamber of the eye by the phacoemulsification needle changes. If this change in volume caused purely by the movement of the needle is also captured, this can more reliably ensure that the control apparatus does not establish dangerous pressure changes in the anterior chamber of the eye on the basis of the movement of the needle.

According to one embodiment of the invention, the measuring device is configured to measure the topography of a cornea, of the eye to be treated by phacoemulsification. In the case of a change in pressure, there is not only a change in the volume of the anterior chamber of the eye, but necessarily also a change in the geometry and contour of the cornea which behaves like a membrane. If both the volume measurement of the anterior chamber of the eye and the measurement of a topography of the cornea are carried out, a statement in respect of a pressure change in the anterior chamber of the eye can be made with even greater reliability.

The measuring device preferably has a keratometer for capturing the topography of the cornea.

In accordance with the invention, the control variable can be used to control preferably a pump power of an aspiration pump, a volumetric flow rate of a fluid for aerating the aspiration line or a valve in the irrigation line. A strong pressure change can therefore foe effectively counteracted by influencing the volumetric flow rates in the irrigation line and/or aspiration line.

### BRIEF DESCRIPTION OF THE DRAWINGS

The invention will, now be described with reference to the drawings wherein:

FIG. 1 is a schematic of an ophthalmic surgical system according to the invention with a control apparatus;

FIG. 2 is a cross-sectional view of an anterior chamber of the eye with an eye lens in the case of a first needle position at the time t₁;

FIG. 3 is a cross-sectional view of an anterior chamber of the eye with an eye lens in the case of a first needle position at a time t₂; and,

FIG. 4 is a cross-sectional view of an anterior chamber of the eye with an eye lens in the case of a second needle position at a time t₂.

### DESCRIPTION OF THE PREFERRED EMBODIMENTS OF THE INVENTION

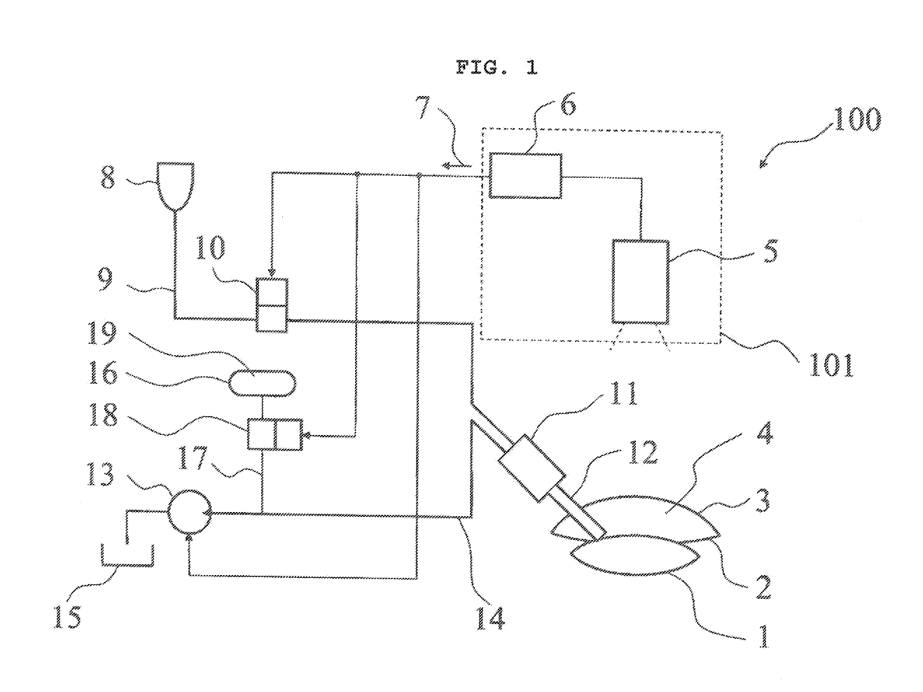

FIG. 1 is a schematic of an ophthalmic surgical system 100 with a control apparatus 101 according to the invention. An iris 2 rests above an eye lens 1 to be treated and, together with a cornea 3 arranged thereover, forms the boundary for an anterior chamber of the eye 4. A measuring device 5 arranged thereover is suitable for capturing the volume of the anterior chamber of the eye 4 at a first time point and at a second time point, wherein the value of this volume is transmitted to a control unit 6 which calculates a control variable 7 therefrom. If an irrigation fluid is routed to a phacoemulsification handpiece 11 having a vibrating needle 12 from a first irrigation fluid container 8 via an irrigation line 9 and a valve 10, the control variable 7 can actuate the valve 10 accordingly in order to avoid further inflow of the irrigation fluid from the irrigation fluid container 8 to the handpiece 11.

An aspiration pump 13 suctions a fluid and appropriately small particles from the anterior chamber of the eye 4 to an aspiration container lb via an aspiration line 14. By way of example, in this case the control variable 7 can act on the aspiration pump 13 in such a manner that the aspiration pump 13 operates with a lower pump power in the case of an occlusion, such that it is not possible for such a strong negative pressure to develop in the aspiration line 14.

If a strong negative pressure is present in the aspiration line in the case of an occlusion and the aspiration line should be vented, the ophthalmic surgical system 100 can also have a second irrigation container 16, from which irrigation fluid 19 can be filled, into the aspiration line 14 through a venting line 17 via a valve 18. The control variable 7 can therefore also actuate the valve 18 in an appropriate fashion such that it is possible to quickly compensate for a negative pressure in the aspiration line 14.

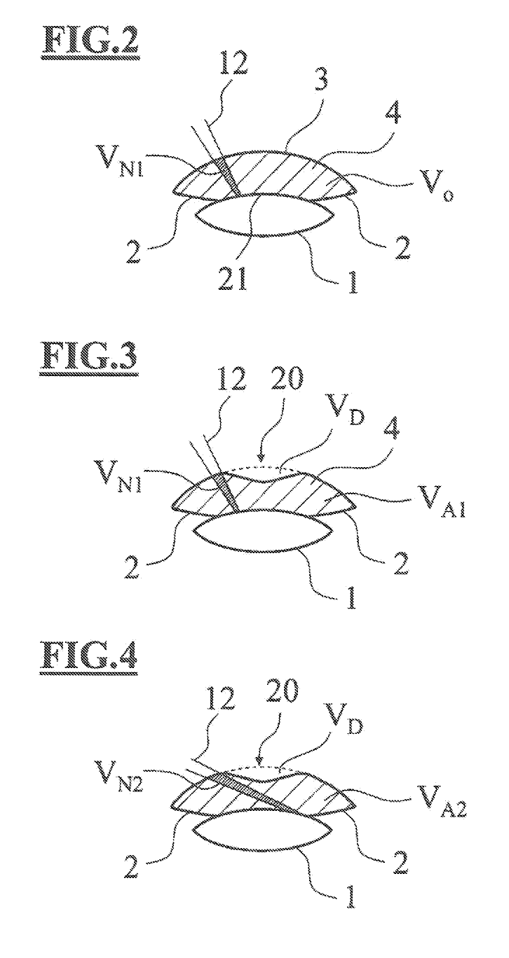

FIG. 2 shows a cross-sectional view of an anterior chamber of the eye 4 with an associated eye lens 1. A needle 12 serves to create small particles of the eye lens 1. The anterior chamber 4 of the eye is formed by an upper part 21 of a lens capsular bag, an iris 2 and a cornea 3. The volume thus formed of the anterior chamber 4 of the eye is denoted by V₀ in FIG. 2. The profile of the cornea 3 is illustrated in convex fashion, with such a profile forming if there is no negative pressure yet in the anterior chamber 4 of the eye. A needle 12 has been inserted in the region of the anterior chamber 4 of the eye, which needle, in this first needle position, requires a volume V_{N1} within the volume V₀ of the anterior chamber 4 of the eye. Hence the volume V_{A1}(t₁) available for a pressure change in an anterior chamber 4 of the eye at the time t₁ can be calculated as follows:

V_{A1}(t₁)=V₀−V_{N1}.

If negative pressure develops in the anterior chamber 4 of the eye in this first needle position, the cornea 3 deforms in such a manner that it loses its continuous convex form and for example arches inward in the center, see reference numeral 20 in FIG. 3. This volume proportion of the anterior chamber 4 of the eye created by a change in pressure is denoted by V_{D} in FIG. 3. As a result, the volume V_{A1} of the anterior chamber of the eye at a time t₂, during a developing negative pressure in the anterior chamber of the eye, is calculated as follows:

V_{A1}(t₂)=V₀−V_{N1}−V_{D}(t₂).

In FIG. 4, a needle is illustrated in a second needle position. In contrast to FIG. 3, the needle 12 has been pushed relatively far into the anterior chamber 4 of the eye, and so it requires a volume V_{N2} within the volume V₀ of the anterior chamber of the eye. The volume V_{A2} of the anterior chamber of the eye is then calculated at a time t₂ as follows:

V_{A2}(t₂)=V₀−V_{N2}−V_{D}(t₂).

Hence, if the measuring device can also capture the part of the needle which is situated within the anterior chamber 4 of the eye, the available eye volume can be calculated more accurately.

If a difference between V_{A1(t}₁) and V_{A1}(t₂) differs from zero, this means that a volume change took place, and so there must have previously been a pressure change in the anterior chamber of the eye. Depending on the value of this difference, the pressure or the volumetric flow rate in an irrigation line 9 and/or aspiration line 14 can subsequently be controlled by the control apparatus according to the invention.

It is understood that the foregoing description is that of the preferred embodiments of the invention and that various changes and modifications may be made thereto without departing from the spirit and scope of the invention as defined in the appended claims.

What is claimed is:

1. A control apparatus for an ophthalmic surgical system for phacoemulsification of an eye to he treated, the eye to be treated including an anterior chamber defining a chamber volume, the ophthalmic surgical system including an aspiration line configured to have a first volumetric flow rate therein and an irrigation line configured to have a second volumetric flow rate therein said control apparatus comprising:
a measuring device configured to determine the chamber volume of the anterior chamber of the eye to be treated at a first time t₁ and at a second time t₂;
a control unit configured to calculate a control variable in dependence upon the volume of the anterior chamber at said, first time t₁ and said second time t₂; and,
said control unit being further configured to apply said control variable to control one of a pressure and a volumetric flow rate in at least one of said irrigation line and said aspiration line.

2. The control apparatus of claim 1, wherein said measuring device has an OCT system configured to measure a three-dimensional object region.

3. The control apparatus of claim 1, wherein the ophthalmic surgical system includes a phacoemulsification handpiece having a needle defining a needle volume, the needle being configured to be inserted into the anterior chamber of the eye to be treated; and, wherein said measuring device is configured to determine the chamber volume of said needle within the anterior chamber.

4. The control apparatus of claim 1, wherein the eye to be treated has a cornea defining a topography, wherein said measuring device is configured to measure the topography of the cornea of the eye to be treated.

5. The control apparatus of claim 4, wherein said measuring device includes a karatometer.

6. The control apparatus of claim 1, wherein the ophthalmic surgical system further includes an aspiration pump having a pump power, the aspiration line is configured to have a fluid flow therein for the aerating of the aspiration line, and the irrigation line has a valve, wherein said control unit is further configured to apply said control variable to control at least one of the pump power of the aspiration pump and the valve of said irrigation line.

7. An ophthalmic surgical system for phacoemulsification of an eye to be created, the eye having an anterior chamber defining a chamber volume, the ophthalmic surgical system comprising:
an aspiration line configured to have a first volumetric flow rate therein;
an irrigation line configured to have a second volumetric flow rate therein said control apparatus comprising:
a measuring device configured to determine the chamber volume of the anterior chamber of the eye to be treated at a first time t₁ and at a second time t₂;
a control unit configured to calculate a control variable in dependence upon the volume of the anterior chamber at said first time t₁ and said second time t₂; and,
said control unit being further configured to apply said control variable to control one of a pressure or the volumetric flow rate in at least one of said irrigation line and said aspiration line.
